(19) United States
(12) Patent Application Publication
(10) Pub. No.: US 2007/0021983 A1
(43) Pub. Date: Jan. 25, 2007

(54) System for healthcare information distribution

(75) Inventor: David Joseph TenBarge, Peapacl, NJ (US)

Correspondence Address: DEBORAH A. BARNES, 3582 SILVERLACE LANE #34, BOYNTON BEACH, FL, 33436, US

(73) Assignee: Blueprint Studios, LLC, Far Hill, NJ (US)

(21) Appl. No.: 11/184,226

(22) Filed: Jul. 19, 2005

(51) Int. Cl. G06F 19/00; G09B 23/28; A61B 5/00

(52) U.S. Cl. 705/3; 434/262; 600/300

(57) ABSTRACT

A system and method of distributing health information through a portal passes through several stages associated with the patients, their ailments and treatments. First, contact and diagnostic data is obtained from the patient, including medical diagnosis, or other information descriptive of the patient's condition. Next, educational information is provided to the patients. Each patient is also asked to identify the treatment his has been prescribed and receives motivational information to induce the patient to either start or continue treatment. Finally, the system also gets involved actively in the treatment by sending messages to the patient requiring certain activities, such as going to see a doctor, exercising, and so forth.

### BACKGROUND OF INVENTION

A. Field of Invention

This invention pertains to a method and apparatus for distributing healthcare information and, optionally, monitoring the health of patients and actively participating in the patient's treatment. Information collated from several patients can be distributed to drug companies, health insurance companies, etc.

B. Description of the Prior Art

Printed health information is widely available from a variety of sources and forms, including books, pamphlets, audiovisual presentations, and so on. The information is sometimes given away but, sometimes the patients pay for it. The information is derived from various sources. Some of the information comes from drug companies or device manufacturers. Other information may come from various governmental agencies or private foundations. Yet other information may come from health care providers, or other organizations. Very often, while the individual or organization providing the information may have a financial or other type of interest associated with the information and therefore the information may be suspect. However, the information recipient, such as a layman reader, may have trouble determining whether a particular source is credible or not.

Health information is also available electronically from a large number of on-line sources as well, usually from the same organizations that also provide the printed health information. The same problems discussed above, are also applicable for this type of information. An additional problem with electronically distributed health information is that the persons benefiting from this information must search it out themselves, since it is very hard for the information providers to identify and target these persons.

Health information presently distributed to (potential) patients and other persons, be it printed, electronic, etc., by its very nature must be fairly generic because it is very difficult to tailor it to specific needs of specific recipients. Moreover, the very act of providing the information is static in the sense, that the information is sent out with either no feedback , or very little feedback to indicate whether anyone has actually received it.

### SUMMARY OF INVENTION

The present invention pertains to a method of distributing health information to several patients, preferably through an Internet portal. Several types of information are provided including educational information, motivational information and activity-related information. A system used to implement the method includes several software based sub-assemblies. One sub-assembly obtains contact and diagnostic information from the client. This information includes data, such as his e-mail address, through which contact is maintained. The diagnostic information includes a description of the patient's ailment and its severity.

The educational information may be somewhat generic but is tied to the diagnosis of the patient. So, for example, for a patient having a high blood pressure, the educational information may include a discussion on the consequences of this condition.

The motivational information is more closely related to the patient and is dependent on specific responses from the patient. For example, if the patient indicates that he is receiving a particular treatment, the motivational information may include a discussion of how successful the treatment has been for other patients with similar conditions.

The activity related information includes messages to the patient requesting that he go for a check up or perform some other activity related to the treatment.

### BRIEF DESCRIPTION OF THE DRAWINGS

FIG. 1 shows a diagrammatic form the functionality and data flow in an information distribution system constructed in accordance with this invention;

FIG. 2 shows a block diagram of a portal providing various health related services to a patient;

FIG. 3 shows a flow chart for a management tool used by the portal of FIG. 2; AND

FIG. 4 shows the flow of information between costumers, a health product supplier and a third party.

### DETAILED DESCRIPTION OF THE INVENTION

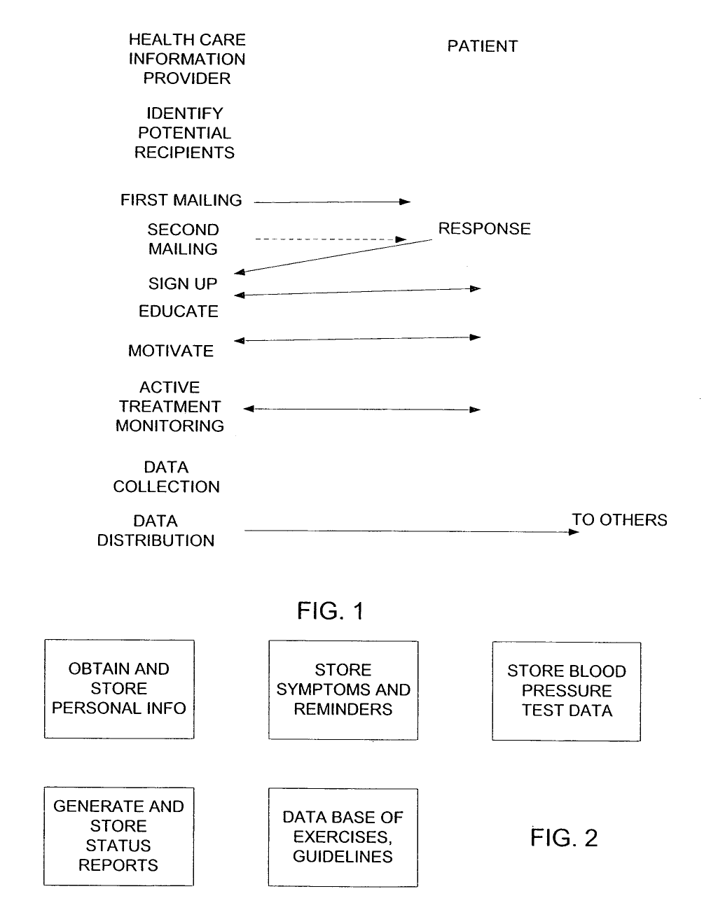

The present invention is a health information distribution system in which health information is provided that is tailored to specific needs of a patient. The information is preferably distributed using the Internet. As shown in FIG. 1, the information distribution is prefaced by a compilation step during which a data base is compiled of addresses of potential patients. (The term ‘patient’is used here to refer to any individual who may be interested receiving information. This person in some instances may be involved in administering treatment, such as taking the blood pressure or administering drug to another person, such as an infant or an invalid). As will be apparent, as part of the process of distributing health information, the system also receives patient information that may be valuable to other organizations. Moreover, the distributing health information may be provided as a service by a health insurance provider.

In any event, during this prefacing step, the names and addresses of patients are collected using various records. For example, if the health information is distributed by, or on behalf of a health care insurer, then the patient data base is culled from the list of subscribers of the insurer.

Once the patient data base is established, a first mailing is sent to these patients. The first mailing is performed using e-mail or regular mail. As indicated in FIG. 1, this step may be repeated at least once, or several times to patients from whom no response is received.

When a response is received from a patient indicating an interest in participating, then a sign up process is initiated during which various information is received that enables the system to provide customized health information as described below. In one embodiment of the invention, the data base identifies only patients having a specific ailment, for example, high blood pressure, and all the provided health information relates to this ailment. In another embodiment, the system is designed to service patients with different ailments.

Once sign up is completed, the system starts distributing different types of health information. Importantly, in many instances, the ‘information’ provided is a message responsive to specific inputs or reactions from the patient so that a dialog is established, as described in more detail below. As shown in FIG. 1, the dialog may have several purposes, including educating the patient, providing motivation to reinforce treatment, and actively assist the patient during his or her treatment. Preferably the dialog is performed through a website or Internet portal.

The portal provides several functions, as shown in FIG. 2. The first function of the portal is to communicate with the patients, as discussed above.

Another function of the portal is to educate the patient. For example, as shown in the Figure, the portal provides some ailment-specific information including diagnosis, as well as other information based on patient characteristics, such as age, gender and race. Moreover, the portal also provides or requests other specific information. For example, the portal may ask the patient if he knows specific data related to his ailment. For example, for high blood pressure therapy, the portal asks the patient if he knows his blood pressure, as well as information on what kind treatment he has been getting if any, and whether he would like to receive any information on the treatment, including any alternate treatments. Each response is recorded in a data file for the patient. In addition, based on the responses, additional information is provided to the patient that reinforces treatment. For example, when the patient first indicates that his problem is high blood pressure, the portal provides to the patient guidelines for exercising, dieting, and/or other information. For example, suggestions from experts can be provided on how often should the patient measure his blood pressure.

If the patient indicates that he does not know his blood pressure, information is provided to the patient to indicate that it is important for the patient to visit his doctor because without active intervention, the patient's condition can become aggravate. Additional information may also be provided including statistics indicating that treatments for high blood pressure are very successful.

If the patient indicates that he is following a prescribed treatment, then, as part of motivation, the portal can provide a discussion of various alternative treatments. If the patient indicates that he is taking some medicine as part of the treatment, the portal can provide a discussion of either alternate medicines, or other discussions, related for example on how the medicine should be taken, why it is important that the patient continue with the treatment, why is it important for the patient to follow the directions provided with the medicine, etc.

Moreover, the portal is also able to take a more proactive role in the patient treatment. For example, the exercise or dieting guides normally describe generic programs that apply to a large number of individuals. Once personal information is obtained about a patient, customized guides may be generate that provide tailored programs for the patient based on sex, age, weight, physical condition and ailment. If the patient decides to take his blood pressure using a home kit, the portal can provide instructions on how to take blood pressure and guide lines on what are the thresholds for the patient, again, based on his individual characteristics.

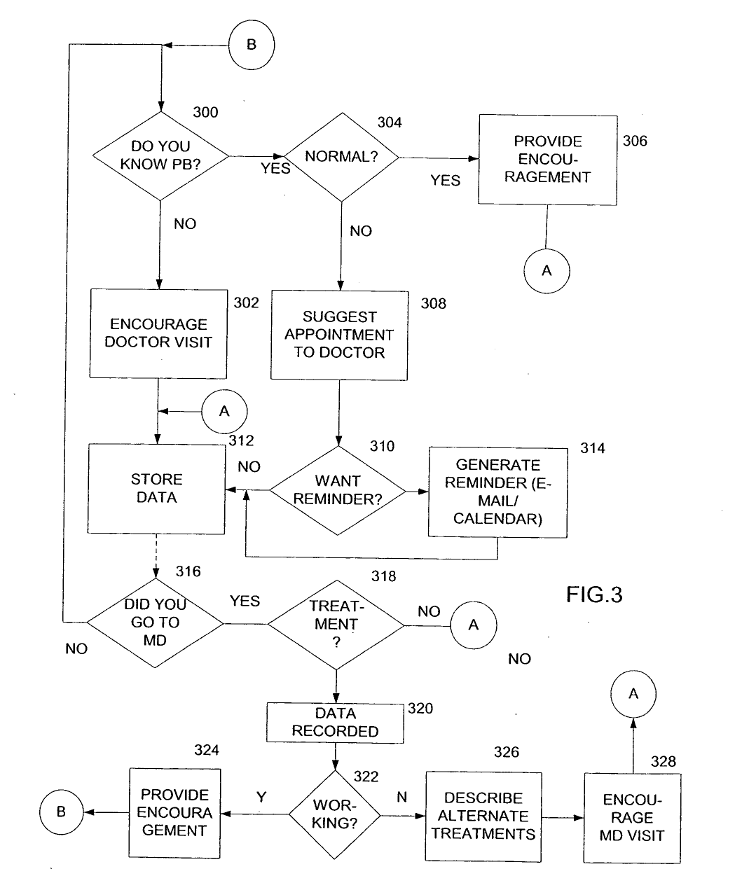

A more detailed description of process is shown in FIG. 3. This process is suited for blood pressure management tool. Similar processes can be used for other ailments. For the sake of clarity, the initial steps of registering the patient and recording patient information have been omitted. In step 300 the patient is asked if he knows his blood pressure. If he does not know it, or if has not seen his doctor for over 6 months, then in step 302 the patient is encouraged to see of physician. For example, as part of this step, the patient may be provided with information indicating the dangers of high blood pressure, hypertension, etc. If the patient indicates in step 300 that he knows his blood pressure, then in step 304 a check is performed to determine if the blood pressure is normal (e.g. less than 80/130). If the blood pressure is normal, in step 306 the patient is encouraged to maintain his blood pressure low. For example, during this step, the patient may be provided with information on what kind of diet and/or exercise is beneficial for this purpose.

If in step 304 it is determined that the blood pressure is not normal, then in step 308 the patient is urged to make an appointment with his doctor. As part of this step, the clinician may be given a list of doctors, if he does not have one. He may also be given information on what to expect during a visit, including what typical tests are to be performed, any prerequisite for the visit (for instance no eating before blood tests), what questions to ask the doctor, etc. In step 310 the patient is also asked if he needs to be reminded to make an appointment, and/or to keep his appointment. If no reminder is requested, then in step 312 the data from the patient is stored. If required, in step 314 reminders are generated and e-mailed (or sent by other means) at the predetermined times to the patient to remind him to make an appointment and/or to remind the patient to go to the doctor on the appointed date. In addition data is again stored in step 312.

The next time the patient signs up, the portal goes directly to step 312 and asks the patient if he did see the doctor. If he did not, then the process goes back to step 300. If he did go to his doctor then in step 318 the portal asks the patient if he has received a treatment. If he has not then, in step 306 the data is stored in step 312 and the patient is encouraged again to visit his doctor.

If he did receive treatment, then the treatment is recorded in step 320. At the same time, information is also provided to the patient describing the advantages and disadvantages of the treatment. For example, if the treatment includes medication, then the patient is informed of potential side effects, and other similar data.

As part of the services provided by the portal, clinical data for the patient is stored. For example, every time the patient takes his blood pressure, or gets his blood pressure measured by a doctor or other health care provider, this measurement is recorded. The clinical data can then be displayed when some predetermined events take place. For example, the clinical data (such the blood pressure for the last six month together with the heart beat) can be displayed every time the patient signs on. Alternatively, the blood pressure can be shown to the patient once a week, or when it reaches certain limits. Of course, this last feature is more valuable if the some clinical data is entered by someone else. For example, the clinical data can be entered into the patient data base by a doctor.

The patient can also be asked in step 322 whether the treatment is working or not. If the treatment is working then in step 324 the patient is provided with encouragement to continue with the treatment. This encouragement may include, for example, a discussion of how often treatment is successful.

If in step 322 the patient indicates that the treatment does not appear to work, then in step 326 the patient is given information on alternate treatments. In step 328 the patient is also encouraged to go back to his doctor and ask about these alternate treatments.

Of course the steps shown in FIG. 3 are illustrative only and the dialog between the portal and the patient can take on many other forms. Moreover, while the dialog shown in the Figure is tailored for blood pressure management, similar dialogs can be provided for other ailments as well.

The portal shown in the Figures can be provided by independent companies, by health care providers, or by health insurance companies. Health insurance companies may find the particularly the portal particularly attractive because it allows them to keep in touch with their customers. Moreover, it is in the interest of the insurance companies to have their customers, e.g., the patients, receive early and effective treatment.

Preferably, the information collected from several patients is collated by the portal. The information collated may include many different factors, such as the reaction, responses and behavior of patients to specific drugs or treatment. Moreover, the portal may also be used to collect other types of information. For example, the portal can collate statistically what treatment and what drugs are being prescribed for a particular set of symptoms. The portal may also be used to record or correlate how different physicians treat various types of ailments, including instances where a particular drug, test or device is prescribed or not prescribed by particular physicians. This type information may be useful to drug companies, equipment manufacturers, physicians, hospitals, etc.

In summary, a health information distribution system is described which exchanges messages with several patients via a portal. This dialog can be categorized into one of four phases. The first phase consists of establishing communication with each patient. As part of this phase, contact information is obtained and stored for each patient. The second phase consists of education. During this phase, the patient's ailment and diagnosis (e.g., the severity) is identified. In the third phase, motivational information is provided to reinforce the patient's will power to obtain or continue with treatment. The fourth phase is an active phase in which specific information tailored to the patient's needs is provided and the patient is urged to take specific actions rather than just providing reinforcing information.

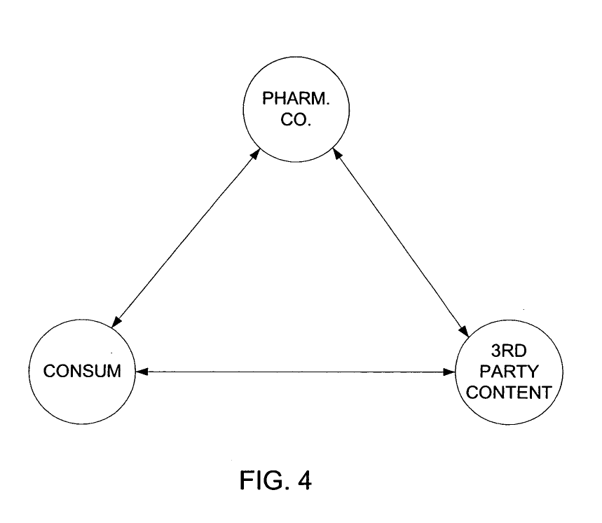

FIG. 4 shows an advantageous arrangement for providing information to targeted costumers using the system described above, and in FIGS. 1-3. The customers provide information to a health care product supplier, such as a pharmaceutical company and/or a third party content provider. The company provides content to the third party about various subjects and materials, generally associated with its products. The third party collects the information from the company and sends it to various costumers in accordance with their needs. For example the third party may be a manage care organization. All the members of the exchange benefit from the exchange of information. The manage care organization provides up to date information to its customers and insures that they get proper and prompt care. In return, it provides patient specific data to the health product supplier. The health product supplier obtains valuable information about the patient/customer and builds up good will with both the manage care organization and the customers. The customers benefit because they receive important information about their health and can also receive information about special pricing programs. As described above, the information exchanged includes education and awareness programming focused on a particular disease or condition and customized to be delivered to specific patients/customers based on data analysis.

Data transfer can be implemented by various means, such as direct communication, electronic newsletters, online website, video on demand, community involvement, at work presentations, and so on. However, the content provided is based on the specific needs of the customers as provided by the manage care organization. The direct communication consists of direct transmission of specific information useful to specific customers. Direct communication may include mailing of printed materials,, or providing materials through video-on-demand using webcasting or local cable channels. This type of presentation may also be sold to an independent company who may then broadcast it for a fee, or as part of a public service offering. Other vehicles can electronic transmissions may include e-news letters, periodicals, e-mail, online chats, newsgroups, and so on. Online transmissions include subject specific web sites. Community involvement includes providing lectures regarding specific illnesses of interest to a specific community. Similar information may be provided at the work-place specifically targeting illnesses associated therewith. Obviously, there is some overlap between these various vehicles.

In one embodiment, the system is operated as follows. Sample data is obtained from the managed care organization. The sample data base is used to build a data base architecture and a data base that is compliant with the requirements related to customers' right to privacy (HIPAA compliant). Direct communication (including mailings of printed material) is implemented using a printing company that is HIPAA compliant. Because of privacy concerns, customers are given the change to opt-in and opt-out of the program as they see fit.

While the invention has been described with reference to several particular embodiments, it is to be understood that these embodiments are merely illustrative of the principles of the invention. Accordingly, the embodiments described in particular should be considered as exemplary, not limiting, with respect to the following claims.

We claim:

1. A method of distributing health information to a plurality of patients comprising:
registering each patient; and
exchanging messages with the patient including requesting diagnostic information from the patient and providing the patient with educational information about his ailment, motivational information regarding his treatment and activity information requesting the patient to take specific acts related to his ailment.

2. The method of claim 1 wherein during said step of exchanging messages, the diagnostic information is checked to determine how serious is the patient's ailment.

3. The method of claim 2 wherein the patient is provided with suggestions to obtain medical help.

4. The method of claim 2 wherein said patient is provided with dieting information.

5. The method of claim 4 wherein said dieting information is tailored to the patient,

6. The method of claim 2 wherein said patient is provided with exercising information,

7. The method of claim 6 wherein said exercising information is tailored to the patient.

8. The method of claim 1 further comprising collating information from the responses of patients.

9. The method of claim 8 wherein said collated information is distributed to other organizations.

10. The method of claim 1 wherein said all the information distribution occurs automatically through a web site.

11. A system for distributing health information to patients comprising:
a portal exchanging messages with a plurality of patients, said portal including subassemblies devoted to provide communication with said patient, educational information, motivational information and activity information.

12. The system of claim 11 wherein said subassembly devoted to communication obtains contact information describing how to send messages to the patient and ailment information describing the patient's ailment.

13. The system of claim 12 wherein said educational subassembly generates and provides information to the patient related to his ailment.

14. The system of claim 12 wherein said motivational subassembly provides motivational information to the patient.

15. The system of claim 14 wherein said motivational subassembly obtains information associated with the treatment recommended to the patient and provides information associated with the treatment.

16. The system of claim 15 wherein said activity subassembly generates messages to the patient to take certain actions associated with the treatment.
